SEQUENCE LISTING (4 sequences)

SEQ ID NO: 1 - DNA, 25 bp, Artificial
  Sequence of Primer 1
gcctcagtcaggcgctcagctccgt

SEQ ID NO: 2 - DNA, 29 bp, Artificial
  Sequence of Primer 2
attggagccccgcacttccaccaccagct

SEQ ID NO: 3 - DNA, 16 bp, Artificial
  Sequence of Primer 3A
agccgccgccgccgcc

SEQ ID NO: 4 - DNA, 17 bp, Artificial
  Sequence of Primer 3B
gcgcggcggcggcggcg

(19) United States
(12) Patent Application Publication
(10) Pub. No.: US 2021/0155981 A1
(43) Pub. Date: May 27, 2021

(54) SYSTEM AND KIT FOR DETECTING THE NUMBER OF CGG REPEATS IN THE 5' UNTRANSLATED REGION OF FMR1 GENE

(71) Applicant: BEIJING MICROREAD GENETICS CO., LTD, Beijing (CN)

(72) Inventors: Chen LI, Beijing (CN); Ye ZHANG, Beijing (CN); Mengjuan HE, Beijing (CN); Miaomiao SHAO, Beijing (CN); Chuguang CHEN, Beijing (CN)

(73) Assignee: BEIJING MICROREAD GENETICS CO., LTD, Beijing (CN)

(21) Appl. No.: 16/693,211

(22) Filed: Nov. 22, 2019

(51) Int. Cl. C12Q 1/6853

(52) U.S. Cl. CPC C12Q 1/6853

(57) ABSTRACT

Disclosed are a detection system and a detection kit for the number of CGG unit repeats in the 5′ untranslated region of genes. The detection system comprises three primers located upstream, downstream and at the boundary of the repeat of CGG repeats, and the number of CGG repeat units can be determined according to the results of the detected full-length product size and number of CGG product. In particular, since the primer located at the boundary of the repeating fragment has stronger binding ability to the corresponding template, the maximum CGG product can be determined more clearly, thereby the specific number of CGG repeats can be accurately determined, the number of repeats which is less than 60 repeats can be effectively and accurately determined, and it is possible to clearly determine whether there is a genotype with a larger number of repeats.

### FIELD

The present invention relates to the detection of the number of CGG repeats in gene, providing a reference for the clinical diagnosis of Fragile X Syndrome, and belongs to the clinical molecular detection technology in the field of biomedicine.

### SEQUENCE LISTING

Sequence Listing is being submitted as an ASCII text file via EFS-Web, file name “190124-Sequence-Listing.txt”, size 1023 bytes, created on Nov. 22, 2019, the content of which is incorporated herein by reference.

### BACKGROUND

Fragile X Syndrome (FXS) is a common X-linked hereditary disease. The typical symptoms are moderate to severe mental retardation, also accompanied by behavioral and physical developmental abnormalities. Its incidence is second only to Down's syndrome in hereditary mental retardation syndromes, accounting for 10%-20% of male mental retardation and 40% of X-linked mental retardation.

The occurrence of Fragile X Syndrome is closely related to the abnormality of the FMR1 gene. More than 95% of the onset of Fragile X Syndrome is caused by the CGG repeat structure expansion mutation in the 5′ untranslated region of the FMR1 gene on X chromosome, and 5% or less is caused by missense mutation and deletion mutation affecting the normal function of the FMR1 gene.

The FMR1 gene is located on chromosome Xq27.3 and has a full-length of 38 kb, containing 17 exons and 16 introns. There is a (CGG), trinucleotide tandem repeat in the 5′ untranslated region of the FMR1 gene. The change in the number n of CGG repeats may affect the CGG repeat region and upstream CpG island methylation, therefore affecting the normal transcription of the FMR1 gene, and then initiating the corresponding clinical symptoms.

According to the number of CGG repeats, the FMR1 gene can be classified into a full mutation, a premutation, an intermediate, and a normal. There are currently two clinically recognized genotype classification standards, which are respectively formulated by the American College of Medical Genetics and the European Society for Human Genetics. The specific numerical values are shown in Table 1.

TABLE 1
FMR1 Genotype Division Criteria Based
on the Number of CGG Repeats

Number of CGG Repeats

| | Guidelines of the | Guidelines of the |
|---|---|---|
| FMR1 | American College of | European Society |
| Genotype | Medical Genetics | for Human Genetics |
| Normal | <45 | <50 |
| Intermediate | 45-54 | 50-58 |
| Premutation | 55-200 | 59-200 |
| Full Mutation | >200 | >200 |

When the number n of CGG repeats is greater than 200, it is defined as a full mutation of the FMR1 gene. Then the CpG island of the FMR1 promoter region is highly methylated, the transcription of the FMR1 gene is inhibited, the protein product is absent, the related neurological functions are affected, and the individual exhibits characteristic features of Fragile X Syndrome such as mental retardation and autism. When the n is between 55-200 or 59-200, it is called a premutation of the FMR1 gene. The premutation produces excess mRNA, which in turn affects the regulation of the expression of multiple proteins. The premutation is considered to be a risk factor causing fragile X-associated Primary Ovarian Insufficiency (FXPOI) and Fragile X-associated Tremor and Ataxia Syndrome (FXTAS).

Fragile X syndrome is a dynamic gene mutation disease. On the basis of recessive inheritance of X chromosome, the number of CGG repeats of the FMR1 gene of the offspring may change based on the number of CGG repeats of the parent. When the number of repeats of the parent is greater than 60, the CGG repeats of the offspring will expand in a certain proportion, the number n of the repeats of the offspring will increase compared to the parent. When the number of the repeats is greater than 100, basically the CGG repeats of the offspring will be expanded, producing more CGG repeats, which may result in a fully mutated FMR1 gene and in turn initiates the Fragile X Syndrome.

The normal FMR1 gene typically has 1-3 AGG insertions within the CGG repeat region. The full mutation and permutation of the FMR1 genes may have no or only a few AGGs. The number of AGGs is believed to be related to the genetic stability of CGG repeats, and the smaller the number of AGGs, the greater the risk of repeat CGG expansion.

The incidence of Fragile X Syndrome is high and the carrier rate is high. There is currently no effective treatment method. It is an effective way to prevent the disease by detecting CGG repeats in the FMR1 gene and reducing the number of the children born with this disease through genetic counseling and prenatal diagnosis in high-risk populations or those with a fertility desire. In particular, female carriers of premutation genes typically have a normal phenotype, while their offspring has a risk of increased CGG repeats. Therefore, in order to detect CGG repeats in the FMR1 gene, it is necessary to detect the premutation based on the detection of the full mutation. In combination with the classification standards of the American Society of Medical Genetics and the European Society of Human Genetics, it is necessary to accurately determine the specific number of 40-60 repeats to meet the need of clinical classification and risk assessment.

Southern blotting is a traditional method for detecting the number of CGG repeats in the FMR1 gene. However, the main limitation of this method is that it is impossible to accurately determine the specific number of CGG repeats, improper operation is likely to produce false negative results, and the operation is cumbersome and is not suitable for large-scale clinical detection.

The number of CGG repeats can be detected by PCR method. However, PCR amplification using only upstream and downstream two primers for routine amplification with a target fragment containing CGG repeats is not suitable for this assay. Since the number of CGG repeats may exceed 1000, excessive CGG repeats mean longer product fragment and higher GC content, which in turn leads to inability to effectively amplify the template, resulting in false negatives. This is especially true for female carrier testing.

For highly repetitive samples with high GC content, researchers have used bisulfite modification to reduce GC content, and then perform PCR amplification to reduce the amplification difficulties caused by high GC. The method has high requirements on DNA template, cumbersome operation, and more critically, the method cannot solve the false negative and expansion difficulty caused by the length of the product fragment.

For the detection of the dynamic mutation diseases including Fragile X Syndrome, repeat-primed PCR (RP PCR) is a relatively effective and recognized method. The method introduces a repeat primer complementary to the repeat sequence to the system, and performs PCR amplification together with the downstream reverse primer. Since the repeat primer may bind to various positions on the repeat region, a series of products in different sizes are produced (as shown in FIG. 1A). When the number of repeats is relatively small, it can be deduced according to the size and quantity of the product; when the number of repeats is relatively large, while the large fragments cannot be efficiently amplified, the smaller fragments of various sizes can be amplified, and their presence suggests the existence of a gene with a high number of repeats, which will avoid false negative results.

One problem of the above method is that since a product comprising relatively long repeats may be used as a template for a relatively short length product, after multiple cycles of PCR amplification, the amount of small fragment products will exponentially exceed the amount of the large fragment products. As a result, the amplification efficiency of the relatively large fragment product is too low, and the number of effective products that can be detected is too small, so the number of repeats cannot be effectively determined. In fact, the original repeated primer PCR method used a total of three primers to overcome this problem (triplet repeat-primed PCR, TP PCR) (Warner et al., J Med Genet, 1996; 33(12): 10022). A heterologous sequence is added at the 5′ end of the repeat primer, the third primer is consistent with this sequence, and the amount of repeat primer is reduced, such that the repeat primer is depleted at an early stage of the PCR amplification, and the subsequent amplification is performed by the reverse primer and the third primer, which avoids the preferential amplification of the short product which depends on the long product, and improves the amplification of the long product (as shown in FIG. 1B).

The products of RP PCR or TP PCR can be detected by agarose electrophoresis, polypropylene gel electrophoresis, capillary electrophoresis, etc. Since the capillary electrophoresis detection has high sensitivity and high resolution, which can quantitatively detect the number of repeats, it is more suitable for such detections and is more widely used.

Compared to dynamic mutation diseases such as Huntington's disease, Fragile X Syndrome is characterized by a large number of repeats of up to 1000; the repeat unit is CGG, with a very high GC content; 40-60 repeats are important for clinical classification, and the specific number of CGG repeats should be accurately detected.

Even with various optimized PCR methods and conditions, repeat fragment products tend to exhibit a decreasing amount of product as the length of the products increases. Due to slippage during PCR, products with more repeats than the actual template may be produced. This will affect the maximum product peak in the repeat product, especially when the number of repeats is relatively large and the peak of the corresponding repeat fragment products is low, such as when the number of CGG repeats is in the range of 40-60 (as shown in FIG. 2A). Some studies or patents (such as Chinese patent CN 102449171 B) add bases matching the specific template sequence at position such as the 3′ end of the repeat primer, the main purpose of which is to more accurately locate the AGG in the CGG repeat region, but it does not solve the problem on determining the maximum product peak in the above-mentioned repeated products.

### SUMMARY

The object of the present disclosure is to provide a system for detecting the number of CGG repeats in the 5′ untranslated region of the FMR1 gene. The detection system combines two methods, full-length PCR amplification of CGG repeat region and repeat-primed PCR (RP PCR), using three primers to perform the amplification to realize the detection of the number of CGG repeats. The number of repeats which are less than 60 repeats can be effectively and accurately determined, and it is possible to clearly determine whether there is a genotype with a larger number of repeats.

The present disclosure also provides a kit for the detection system.

A primer composition for amplifying CGG repeats in the 5′ untranslated region of the FMR1 gene, comprising at least three primers: a primer 1 located upstream of the CGG repeats, a primer 2 located downstream of the CGG repeats and a primer 3 located at the boundary of the CGG repeats. The “boundary” refers to a region comprising part of CGG repeats and part of genomic sequence.

The primer 3 comprises:

(a) at the 3′ end of the primer, 9, 10, 11, 12, 13, 14, 15, 16, 17 or 18 nt containing GCG or GCC repeats; and

(b) at the 5′ end adjacent to the 3′ repeat sequence, 1, 2, 3, 4, 5 or 6 nt identical to the corresponding region of GGCAGC or GGCCCA.

The gene is the FMR1 gene, and the primers are respectively:

preferably, primer 3:
AGCCGCCGCCGCCGCC
or
GCGCGGCGGCGGCGGCG;
preferably, primer 1:
GCCTCAGTCAGGCGCTCAGCTCCGT;
primer 2:
ATTGGAGCCCCGCACTTCCACCACCAGCT.

A modification is provided or a normal base is replaced with a modified base in any of the primer 1, 2 and 3. For example, the modification may be selected from the group consisting of fluorescent group modification, phosphorylation modification, thiophosphorylation modification, locked nucleic acid modification, or peptide nucleic acid modification.

1, 2 or 3 bases at the 3′ end -2 to -15 positions of the primers 1, 2 or 3 are altered, and/or the sequences after the -15 position at the 3′ end of the primers are altered; and the alterations is selected from the group consisting of the addition, deletion and/or substitution of one or more nucleotides. The position of the last nucleotide at the 3′ end is defined as -1 position.

The amplification is performed simultaneously in one amplification system or separately in two or more systems.

The amplification is performed separately in two systems; in a first system, the primer 1 and the primer 2 are used for amplifying to obtain a full-length product; in a second system, the primer 3 and primer 1 or primer 2 complementary to the sequence on the other side of CGG repeats are used to obtain CGG products. The “full-length product” refers to a product containing the whole CGG repeats region, and the “CGG products” refer to products containing different copy numbers of CGG.

A method for determining the number of CGG repeats in the 5′ untranslated region of the FMR1 gene is provided. The method uses the above-mentioned primer composition for amplification to detect the sizes and amounts of CGG products, and the size and amount of full-length product, and then combines these two results to determine the number of CGG repeats. When the numbers of repeats inferred from the two results are consistent, a clear determination is made on the specific number of CGG repeats; when the two results are inconsistent, especially when the number of CGG repeats of the CGG product is greater than the number of CGG repeats corresponding to the full-length product size, it is determined that the sample has a high CGG repeats number in the FMR1 gene.

A kit for detecting the number of CGG repeats in the 5′ untranslated region of the FMR1 gene includes the primer composition of any of the above.

The gene is the FMR1 gene, and the primers are respectively:

primer 1:
GCCTCAGTCAGGCGCTCAGCTCCGT,
primer 2:
ATTGGAGCCCCGCACTTCCACCACCAGCT,
primer 3:
AGCCGCCGCCGCCGCC.

The primers used in the present disclosure include: a primer 1 located upstream of CGG repeats, a primer 2 located downstream of CGG repeats, a primer 3 at the boundary of CGG repeats (as shown in FIG. 1C).

One of the most important innovations of the provided method is that the repeat primer is complementary to the CGG boundary sequence (as shown in FIG. 1C).

With such a design, the repeat primer can still rely on its 3′ sequence to bind to each position on the repeat fragment to initiate amplification. At the same time, since the repeat primer is complementary to the CGG boundary sequence, the matching bases to the boundary region are more than those to the internal repeat sequence, so that the binding ability of the repeat primer is stronger, and the amplification efficiency is higher. It then makes, first, the amplification product corresponding to the maximum number of repeats has higher amplification efficiency in the system than other repeat products, the product amount is more than other products, and it is easier to determine the repeat product corresponding to the maximum number of repeats, and eliminate various interference caused by amplification slippage and the like; second, the ratio of the relatively short repeat fragment amplification products in the total product is relatively reduced, increasing the ability to efficiently amplify repeat product with a larger number of repeats, even in the condition that the maximum repeat product cannot be amplified.

The provided method detects the amounts of CGG products, and the size and amount of full-length product at the same time, then these two results are combined to determine the number of CGG repeats.

As described above, the detection of the number of CGG repeats can also be achieved based on the amounts of CGG products or the size and amount of the full-length product alone, but has defects if used as a clinical detection method. Based on the sizes and amounts of CGG products alone, when the number of repeats is very high or even slightly high (greater than 40), it is difficult to clearly determine the number of repeats; based on the size and amount of the full-length product alone, it is impossible to differentiate normal homozygous samples and full mutation/premutation heterozygous samples, and will cause false negatives. Combining the two results to determine the number of CGG repeats will avoid the above defects, and the reliability of the detection is increased. In the condition of small repeat numbers, the two test results corroborate with each other; in the condition of middle repeat numbers, since the repeat primer is complementary to CGG boundary sequence, the results of CGG products can more clearly determine the number of repeats, and corroborate with the full-length results; in the condition of large repeat numbers, when the CGG repeats number of the CGG product is greater than the CGG repeats number corresponding to the full-length product size, it is determined that the sample has a high CGG repeats number, thus effectively avoiding false negatives.

The present disclosure also provides a kit for detecting the number of CGG repeats in the 5′ untranslated region of the FMR1 gene based on the aforementioned method.

The provided kit uses the aforementioned detection method and detection strategies. The kit comprises a primer composition, an enzyme complex, an amplification buffer system or a mixture of the above components, and further includes components such as known repeat number control, capillary electrophoresis detection related reagents.

The use of the provided kit mainly includes the following steps: amplification system preparation; PCR amplification; capillary electrophoresis; data analysis.

The provided kit can effectively and accurately determine the number of repeats which is less than 60 repeats, and determine whether there is a genotype with a larger repeat number. In addition, it has the characteristics of simple operation, high specificity, high sensitivity, high throughput, high reliability and low cost.

Although the method of the present disclosure is used for detecting the number of CGG repeats in the 5′ untranslated region of the FMR1 gene, it can be applied to the detection of the number of CGG repeats in the 5′ untranslated region of any gene.

### BRIEF DESCRIPTION OF DRAWINGS

FIG. 1: Schematic diagram of primer design for various methods for detecting the number of CGG repeats. The boxed area is the CGG repeat region, and the arrow represents the primer used for the detection and the corresponding position.

A. Repeat-primed PCR (RP PCR) primer design. Repeat primers can bind to various positions on the repeat fragment, so a serious of products in difference sizes will be produced;

B. Triplet repeat-primed PCR (TP PCR) primer design. A heterologous sequence is added at the 5′ end of the repeat primer, and the third primer is corresponding to this sequence;

C. The primer design of the present disclosure. A sequence complementary to the CGG boundary sequence is added at the 5′ end of the repeat primer (as shown in the hollowed box), the repeat primer can still bind to various positions on the repeat fragment; when it binds to the CGG boundary, the matching sequence is longer.

FIG. 2: Comparison of repeated PCR detection results corresponding to repeat primers matching different lengths of CGG boundary sequence. In the condition that the complementary base of the CGG boundary sequence is added at the 5′ end of the repeat primer with 0nt(A), 1nt(B) or 3nt(C), the result shows the repeated fragment PCR detection performed on a female sample with a CGG repeat number of 30/55. The arrows indicate the repeat product peaks corresponding to 30CGG and 55CGG.

FIG. 3: detection results of samples with different numbers of CGG repeats. The kit of the present invention is used to test different samples with, including full-length products and repeat products.

A, a heterozygous sample with a full mutation and 30 CGG repeats; B, a heterozygous premutation sample with 58 and 30 CGG repeats; C, a normal sample with 29 and 30 CGG repeats. The arrows indicate the repeat product peaks corresponding to the repeat number of the sample.

### DETAILED DESCRIPTION

The detection of the number of CGG repeats in the 5′ untranslated region of the FMR1 gene is only taken as an example below. The embodiments are merely for illustration of the effectiveness of the method and would not limit it.

### Example 1: CGG Repeats Detection Using Repeated Primers with Different Lengths of Complementary Fragments to CGG Boundary Sequence

The following three primers were used as repeat primers to detect CGG repeats of the sample:

Primer A:
GCCGCCGCCGCCGCC
Primer B:
AGCCGCCGCCGCCGCC
Primer C:
CCAGCCGCCGCCGCCGCC

The 3′ ends of the three sequences are identical and are all complementary to 5 (CGG)s. The difference is that primer A contains only the repeat fragment sequence, without the sequence complementary to the CGG boundary sequence, which is corresponding to the primer used in conventional repeat-primed PCR (RP PCR); one base complementary to the CGG boundary sequence is added upstream to the 5′ end of the repeat fragment of Primer B; three bases complementary to the CGG boundary sequence is added upstream to the 5′ end of the repeat fragment of Primer C.

The sequence of the upstream primer was: FAM-GCCTCAGTCAGGCGCTCAGCTCCGT.

In addition to primers, the amplification system also included the following components: DNA polymerase (AptaTaq, Roche); amplification buffer (Suzhou MicroRead Technology Co., Ltd.), including dNTPs, 7-deaza-dGTP, betaine, etc.

The tested sample was a female sample with a CGG repeat number of 30/55.

The specific detection steps are as follows:

1) Preparation of PCR amplification reaction system. Each amplification reaction system included 5 μl of primer mixture, 10 μl of amplification buffer, 1 μl of DNA polymerase, 1 μl of sample DNA to be tested, and supplemented with sterile water to 20 μl.

2) PCR amplification. The reaction conditions were: 95° C., 5 minutes; 30 cycles of 94° C., 30 seconds, 60° C., 30 seconds, 72° C., 2 minutes; 60° C., 30 minutes.

3) Amplification products were subjected to capillary electrophoresis. A sample mixture containing molecular weight internal lane standard and formamide (0.5 μl of molecular weight internal lane standard+8.5 μl of formamide) was prepared; 1 μl of amplification product was added to 9 μl of the sample mixture and mixed well, the mixture was subjected to denaturation at 95° C. for 3 minutes and ice bath for 3 minutes. The detection was performed following the steps in the Genetic Analyzer User Manual. The test is recommended to set as that the injection time is 10 seconds, the injection voltage is 3 kV, and the run time is 1,800 seconds.

4) Data analysis. Related files were imported into the software GeneMapper, including Panel, Bin, corresponding Analysis Method, and ROX500 internal lane standard. Sample data source was entered (.fsa file), the previously imported files in the relevant parameter selection field were selected, data was analyzed.

The final electrophoresis results are shown in FIG. 2. Wherein Figure A is the result using the repeat primer A, Figure B is the result using the repeat primer B, and Figure C is the result using the repeat primer C.

As shown, the results of the detection using different repeat primers are generally similar. The amplification products consisted of a series of products 3 nt different from each other, corresponding to the products generated by the binding of repeat primers to different positions of the CGG repeat region. Wherein the smallest fragment of the repeat products corresponded to 5 CGG repeats, after which the next peak of each 3 nt larger corresponds to the amplification product with an additional CGG repeat. Since the relatively long product may be used as a template for generating the relatively short product in the amplification, as the peak height indicating the amount of the product, there is a decreasing tendency for the small fragment peaks to be higher and the large fragment peaks to be lower. In addition, because of the AGG insertion in the CGG repeat region, some product peaks are missing or the peak height is significantly reduced, usually 5 consecutive product peaks. According to the peak shape, it can be determined that the CGG repeats of these two FMR1 copies of the sample are: (CGG)₉AGG(CGG)₉AGG(CGG)₁₀ and (CGG)₄₄AGG(CGG)₁₀. The detection of AGG is not claimed in the present invention, and therefore will not be further discussed herein.

Since the peak height of the repeat product decreases as the length of the fragment increases, and the AGG interference may exist, it is difficult to determine the product peak with the maximum length for the sample with a relatively large number of repeats, i.e., to accurately determine the number of CGG repeats. As shown in FIG. 2A, the arrow on the right side shows the repeat product peak corresponding to the 55 repeats, but there is no significant difference between its peak height and the peak height of the several adjacent product peaks, so it is difficult to be accurately recognized. In fact, for the repeat product peak corresponding to the 30 repeats indicated by the arrow on the left, due to the interference of another different copy of the amplification product, it is difficult to recognize it by a less experienced person. If it is difficult to accurately determine the maximum product peak, then it is impossible to accurately determine the number of CGG repeats, which will have a great impact on the clinical diagnosis application, especially when the number of CGG repeats is in the 40-60 repeat interval, which will directly lead to the inability to determine the full mutation, the premutation and normal sample.

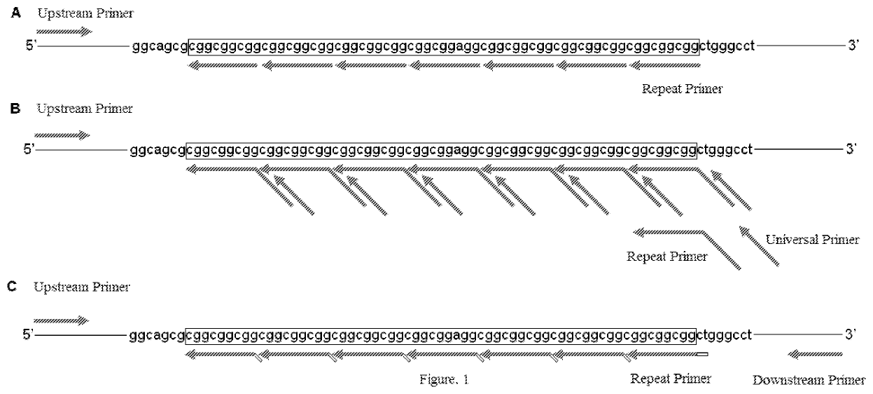

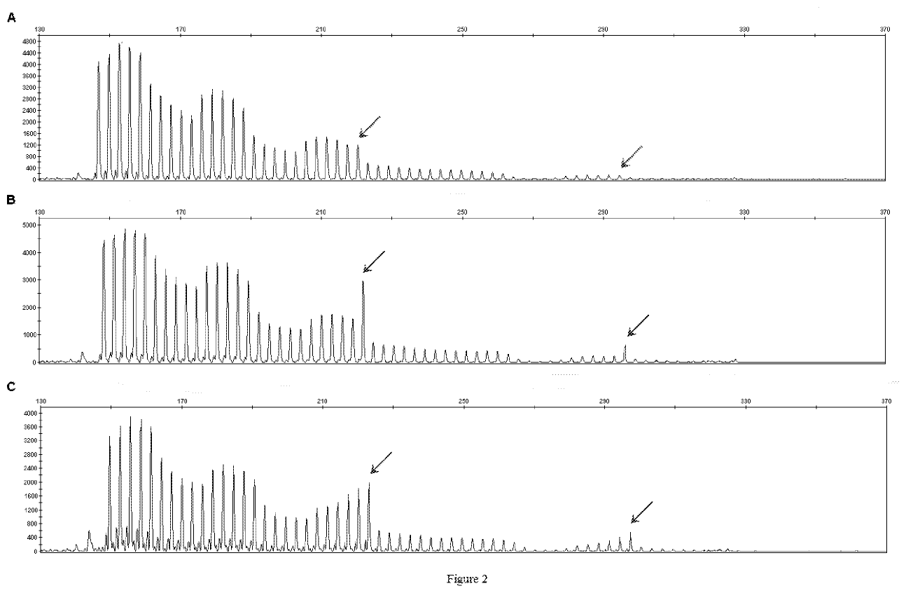

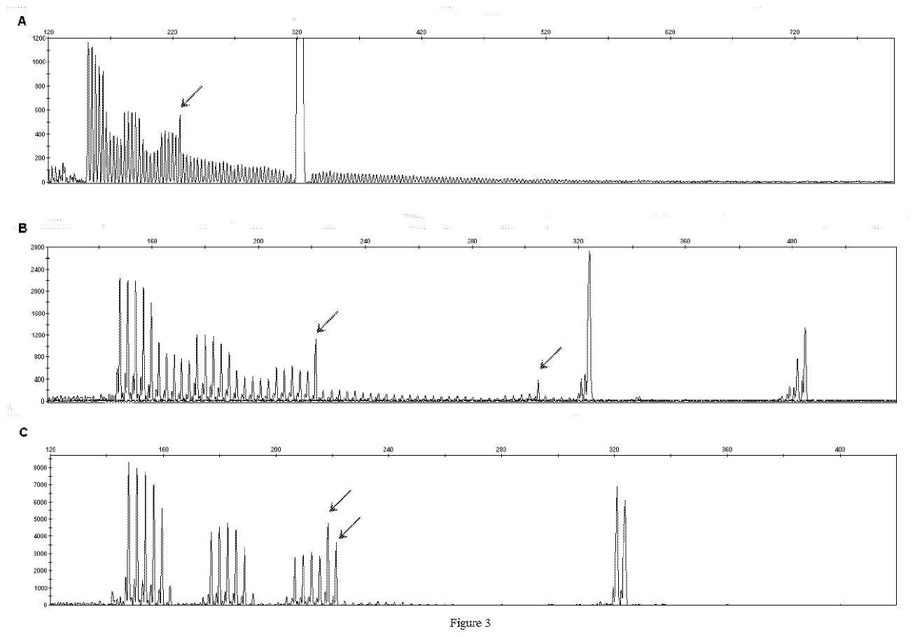

The above problem that it is difficult to accurately determine the maximum length product peak was well improved when using the repeat primer B. As shown in FIG. 2B, the two product peaks corresponding to the 30 and 55 repeats indicated by the arrows have peak heights significantly higher than the adjacent product peaks. For the 55 repeats product, the peak height is 5 times higher than that of the adjacent product peaks; for the 30 repeats product, although there is an interference of another copy, the peak height can reach twice as high as the peak of the adjacent products. Such a difference makes it possible to determine the maximum length product peak very simply and clearly, and thereby determining the number of CGG repeats of the sample. The above difference is due to the base G added at the 3′ end of the repeat fragment of the primer B, so that the repeat primer B can be completely complementary to the CGG boundary sequence (as shown in FIG. 1C). Thus, the binding length of the repeat primer B to the CGG boundary region is more than that to the internal repeat sequence, so the binding ability is stronger, and the amplification efficiency is higher. This amplification advantage is further amplified with the PCR cycles. Finally, the product amount of the product corresponding to the maximum number of repeats is greater than that of other products, and it is easier to determine the corresponding repeat product of the maximum number of repeats.

When using a repeat primer C that increases 3 nt complementation, as shown in FIG. 2C, the product peaks corresponding to the 30 and 55 repeats indicated by the arrows were also significantly improved. Since there are more matching bases and the amplification efficiency is higher when binding to the CGG boundary, it indirectly increased the peak height of the adjacent products with fewer repeats at the same time when increasing the maximum peak height of the product. As a result of this, although the maximum product peak can be determined relatively obviously, the difference is not as significant as that in FIG. 1B.

In summary, it is difficult to determine the maximum length product peak by using the repeat primer alone (repeat primer A); using a repeat primer with a fragment complementary to the CGG boundary sequence at the 3′ end can increase the peak height of the maximum length product peak, so that the maximum product peak can be clearly and accurately determine; as a preference, the different effect of the repeat primer B of which one matching base is added at the 3′ end of the repeat fragment is most desirable.

### Example 2: Detection of Different Types of Samples Using the Kit of the Present Disclosure

The kit components included: enzyme mixture, full-length primer mixture, repeat primer mixture, amplification buffer, positive control, sterile water, internal lane standard, etc.

The specific detection steps are as follows:

1) Preparation of PCR amplification reaction system. Each amplification reaction system included 2.5 μl of full-length primer mixture, 2.5 μl of repeat primer mixture, 10 μl of amplification buffer, 1 μl of DNA polymerase, 1 μl of sample DNA, and supplemented with sterile water to 20 μl.

2) PCR amplification. The reaction conditions were: 95° C., 5 minutes; 30 cycles of 94° C., 30 seconds, 60° C., 30 seconds, 72° C., 4 minutes; 60° C., 30 minutes.

The subsequent detection steps were the same as those in Example 1.

The main difference between using the kit and Example 1 is that the kit provides three primers, an upstream primer, a repeat primer and a downstream primer, and the full-length fragment is amplified while the repeat fragment is amplified, the number of repeats was determined based on a combination of the full-length product and the repeat product results.

The sequence of the upstream prime was: FAM-GCCTCAGTCAGGCGCTCAGCTCCGT;

the sequence of the repeat primer was: AGCCGCCGCCGCCGCC;

the sequence of the downstream primer was: ATTGGAGCCCCGCACTTCCACCACCAGCT;

As shown in FIG. 3, the product peak of which the peak shape, peak height, and tendency are significantly different from those of the repeat product peaks in the range of about 300 nt and greater is the full-length product peak. Using the product size of a full-length product peak with a known number of repeats, a fitting equation for the product size and the number of repeats can be obtained, whereby the number of CGG repeats corresponding to the full-length product can be deduced. This method is more accurate when the number of repeat is relatively small, but may have a certain deviation when the number of repeats is too large.

The following is a detailed description of the specific method for the determination of the number of repeats based on a combination of the full-length product and the repeat products according to three actual test results.

The test result of sample 1 is shown in FIG. 3A. A very high full-length product peak can be observed at about 330 nt, and the corresponding number of repeats was deduced to be about 30 according to the fragment size. The repeat product peaks are a series of consecutive peaks with an overall decreasing tendency, and there is a product peak with a significantly elevated peak height (as indicated by the arrow) at 230 nt. It is the 26^{th} product peak, i.e. the corresponding number of repeats is 30, which is consistent with the corresponding result of the full-length product. There is still a large number of consecutive decreasing product peaks in the larger fragment interval of the product peak, extending at least to 500 nt, and the maximum product peak cannot be determined. These product peaks indicate that there was also a copy of the FMR1 gene whose number of repeats was too large to be effectively detected. Based on the 500 nt product peak, the number of repeats is deduced to be more than 120. Combining the results of the repeat products and the full-length product, the sample was a heterozygous sample with a 30 repeats and a high number of repeats. If there was only a full-length result, the sample would be determined to be a 30 repeat homozygous female or a 30 repeat male, which would result in false negatives. It is also the necessity to determine it based on a combination of full-length and repeat results.

The test result of sample 2 is as shown in FIG. 3B. Two full-length product peaks can be observed at about 330 nt and 420 nt, and the corresponding numbers of repeats were deduced to be about 30 and about 60 according to the fragment size. The repeat product peaks are a series of consecutive peaks with an overall decreasing tendency, with two maximum product peaks (as indicated by the arrows) clearly visible. They are the 26^{th} and 54^{th} product peaks, respectively, with corresponding numbers of repeats of 30 and 58, which are consistent with the corresponding results of the full-length product. There is no large number of consecutive decreasing product peaks similar to FIG. 3A within the larger fragment interval, showing that there is no other FMR1 copy with high repeat number. Combining repeat products and full-length product results, the sample was a heterozygous sample of a 30 repeats and a 58 repeats, clinically classified as a premutation heterozygote. If based on the full-length product result alone, it would be difficult to determine the specific number of repeats for the 58 repeats. Although fitting the equation by increasing the data of different sample sizes can increase the accuracy of the fitting equation, since the electrophoretic mobility has certain differences between different instruments, it is necessary to correct each instrument or even each test, which will greatly increase the workload and detection costs. Moreover, the full-length product peak of larger numbers of repeats is usually a cluster, and it is difficult to accurately determine the true product peak. Combining the repeat product result based on the full-length result makes it simple and clear to determine the number of repeats, since the number of repeats is quantized, no deducing, such as fitting, is needed, an accurate number of repeats can be directly obtained. The innovative “repeat primer complementary to the CGG boundary sequence” used by the present disclosure can significantly improve the maximum product peak differentiation index, and plays a key role in accurately determining the number of repeats.

The test result of sample 3 is shown in FIG. 3C. Two full-length product peaks can be observed at about 320-330 nt, and the corresponding number of repeats was deduced to be about 30 according to the fragment size, which was different from each other by one repeat. The repeat product peaks are a series of consecutive peaks with an overall decreasing tendency, with two maximum product peaks (as indicated by the arrows) clearly visible. They are the 25^{th} and 26^{th} product peaks, respectively, with corresponding numbers of repeats of 29 and 30, which are consistent with the corresponding results of the full-length product. There is no large number of consecutive decreasing product peaks similar to FIG. 3A within the larger fragment interval, indicating that there is no other FMR1 copy with high repeat number. Combining repeat product and full-length product results, the sample was a heterozygous sample of a 29 repeats and a 30 repeats, clinically classified as a normal.

What is claimed is:

1. A primer composition for amplifying CGG repeats in the 5′ untranslated region of the FMR1 gene, comprising at least three primers: a primer 1 located upstream of the CGG repeats region, a primer 2 located downstream of the CGG repeats region and a primer 3 located at the boundary of the CGG repeats region.

2. The primer composition according to claim 1, wherein the primer 3 comprises:
(a) at the 3′ end of the primer, 9, 10, 11, 12, 13, 14, 15, 16, 17 or 18 nt containing GCG or GCC repeats; and
(b) at the 5′ end adjacent to the 3′ repeat sequence, 1, 2, 3, 4, 5 or 6 nt identical to the corresponding region of GGCAGC or GGCCCA.

3. The primer composition according to claim 2, wherein the sequence of the primer 3 is AGCCGCCGCCGCCGCCorGCGCGGCGGCGGCGGCG.

4. The primer composition according to claim 1, wherein,
the sequence of the primer 1 is GCCTCAGTCAGGCGCTCAGCTCCGT; and
the sequence of the primer 2 is ATTGGAGCCCCGCACTTCCACCACCAGCT.

5. The primer composition according to claim 1, wherein a modification is provided or a normal base is replaced with a modified base in any of the primer 1, 2 and 3.

6. The primer composition according to claim 5, wherein the modification is selected from the group consisting of fluorescent group modification, phosphorylation modification, thiophosphorylation modification, locked nucleic acid modification, and peptide nucleic acid modification.

7. The primer composition according to claim 1, wherein 1, 2 or 3 bases at the 3′ end -2 to -15 positions of the primers 1, 2 or 3 are altered, and/or the sequences after the -15 position at the 3′ end of the primers are altered; and the alterations is selected from the group consisting of the addition, deletion and/or substitution of one or more nucleotides.

8. The primer composition according to claim 1, wherein the amplification is performed simultaneously in a single amplification system or separately in two amplification systems.

9. The primer composition according to claim 1, wherein the amplification is performed separately in two systems; in a first system, the primer 1 and the primer 2 are used for amplifying to obtain a full-length product; in a second system, the primer 3 and primer 1 or primer 2 complementary to the sequence on the opposite side of CGG repeats are used to obtain CGG repeat products.

10. A method for determining the number of CGG repeats in the 5′ untranslated region of the FMR1 gene in a sample, comprising performing the amplification using the primer composition according to claim 1, detecting the CGG products and the full-length product, and determining the number of CGG repeats by analyzing the two detection results of the CGG products and the full-length products; if the numbers of repeats inferred from the two detection results are consistent, a clear determination is made on the specific number of CGG repeats; if the two detection results are inconsistent, especially if the number of CGG repeats of the CGG products is greater than the number of CGG repeats corresponding to the full-length product size, it is determined that the sample has a high CGG repeats number in the FMR1 gene.

11. A kit for detecting the number of CGG repeats in the 5′ untranslated region of the FMR1 gene, comprising the primer composition according to claim 1.
